(19) United States
(12) Patent Application Publication
(10) Pub. No.: US 2025/0024793 A1
(43) Pub. Date: Jan. 23, 2025

(54) Use of Frass, Protein and Chitin of Coleoptera and Orthoptera Insects in the Cultivation of Edible and Medicinal Mushrooms in a Symbiotic Closed-Loop Production System

(71) Applicant: KONRAD WLODARCZYK, MASLOMIACA (PL)

(72) Inventor: KONRAD WLODARCZYK, MASLOMIACA (PL)

(21) Appl. No.: 18/776,452

(22) Filed: Jul. 18, 2024

Related U.S. Application Data
(60) Provisional application No. 63/527,345, filed on Jul. 18, 2023.

(51) Int. Cl. A01G 18/20; C05F 3/00; C05F 5/00; C05F 11/02

(52) U.S. Cl. CPC A01G 18/20; C05F 3/00; C05F 5/002; C05F 11/02

(57) ABSTRACT

Insect frass used in combination with mushroom cultivation provides superior results such as increased yields and less greenhouse gas release relative to mushroom cultivation that takes place in the absence of insect frass. The use of insect frass during mushroom cultivation can provide synergistically improved outcomes relative to mushroom cultivation or the production of insects and insect frass in the absence of the other. By using mushrooms and/or their byproducts and insect frass and its components in combination, one can generate a closed loop system that will provide beneficial outcomes to mankind including but not limited to reduced greenhouse gases, greater employment opportunities, synergistic effects related to improved protein and other nutrient production and yields, and/or higher mushroom yields.

This application claims priority under 35 USC 119 (e) to U.S. Provisional Application No. 63/527,345 filed Jul. 18, 2023, the entire content of which is incorporated by reference in its entirety.

### FIELD OF THE INVENTION

The present invention relates to using insect fertilizer (e.g., frass) as well as protein and chitin from the production of various insects to cultivate mushrooms. In a variation, the present invention relates to using the frass, protein, and chitin from the production of Coleoptera (in particular the Alphitobius diaperinus) and Orthoptera insects on an industrial scale in the cultivation of edible and medicinal mushrooms (in particular the Agaricus bisporus).

### BACKGROUND OF THE INVENTION

World production of mushrooms will reach 24 million tons per year in 2027. See, for example, https://www.marketdataforecast.com/market-reports/button-mushrooms-market, https://www.fortunebusinessinsights.com/industry-reports/mushroom-market-100197.

In Europe, the edible mushroom market is expected to expand in sales from EUR 18.2 billion in 2023 to EUR 30.93 billion by 2029, thereby growing at a CAGR (compounded annual growth rate) of 9.23% from 2024 to 2029. See https://www.gombaforum.hu/en/2023/economy/mushroom-consumption-campaign-launches-in-9-european-countries/. Most of this prevailing demand is satisfied by EU-based producers with half of the potential being realized by Poland and the Netherlands, covering over 50% of the total European production. With some 2,900 producers in Europe, the sector is also very important in terms of employment, providing more than 40,000 direct jobs, and many indirect jobs, with many of these employment opportunities occurring in rural areas.

In addition to the cultivation of well-known species of mushrooms in a traditional way for the needs of food consumption, the dynamically developing production of mushrooms as meat substitutes and less popular species for the medical and pharmaceutical market is also gaining new importance. See, for example, https://www.expertmarketresearch.com/reports/medicinal-mushroom-market.

In Europe and North America, the main cultivated mushroom is Agaricus bisporus. For every kilogram of ready-to-eat mushrooms, there is between 2 to 4 kg of compost and casing soil, the production process of which is long, complicated and carbon-intensive, also in the waste disposal phase.

Substrate production takes place in 3 main phases:
Phase I comprises using (depending on technology) wheat straw bedding containing horse manure, hay, corn cobs, cottonseed hulls, poultry manure, brewer's grain, cottonseed meal, cocoa bean hulls and/or gypsum, water and liquid manure to create compost for growing mushrooms, with manure constituting about 50% of the mass of the mixture. The production of phase I compost lasts about 14 days, during which the cold phase (about 3 days at 56° C.) and the so-called hot phase of sanitization takes place in which caramelization occurs, lasting for 10 days at a temperature that is at a minimum of 85° C. After cooling, phase II begins. Phase II comprises undergoing pasteurization and maturation to develop selective microflora for mushroom cultivation. This process typically lasts about 7 days in aerobic conditions until ammonia is released, which is harmful to mushrooms. It proceeds in two phases: for 4-6 hours at 65° C. (pasteurization) and then for 3-4 days at 48° C. After cooling, the mycelium is sown into the prepared substrate and placed in overgrowth tunnels for 16 days. Subsequently, phase III begins.
Phase III comprises removing the overgrowth tunnels, and placing the substrate on shelves or boxes where the mushrooms are cultivated.

When the substrate is placed on boxes or shelves, sometimes feed additives are introduced, which include, among other ingredients, high amounts of protein and/or chitin. A cover consisting of raised peat is then applied at the surface to maintain a suitable level of moisture throughout the cultivation period.

Subsequently, the actual cultivation and harvesting occur in one, two, three or even four flushes.

The cultivation of arboreal mushrooms is different. They are cultivated by inoculating mycelium into bales of various sizes, the bales comprising primarily the shavings of deciduous trees, straw, fruit tree sawdust, cereals such as wheat, corn, soy hull, sorghum, and/or their milling products such as bran and/or even nut shells, hemp straw, coconut, vermiculite and/or cardboard. These bales are poor in nutrients and often insufficient to produce adequate growth. The following species are mainly cultivated in this way: oyster mushrooms (Pleurotus ostreatus, Pleurotus eryngii), shiitake (Lentinula edodes), pioppino (Agrocybe aegerita), Lion's Mane (Hericium erinaceus), Buna shimeji (Hypsizygus tessulatus), and enoki (Flammulina velutipes). Because the harvesting of arboreal mushrooms is usually done in a single flush, the quantity and size of bales needed is huge. For example in the cultivation of Lion's Mane, 1.5-2 kg of bale is needed to produce 600-800 g of fruit bodies each flush.

Insect farming and breeding on an industrial scale is one of the most dynamically developing new branches of agricultural production and the biotechnology industry. According to a Rabobank analysis, the global production of edible insects is expected to increase fifty-fold to 500,000 tons of insect protein in 2030. See, for example, https://www.aquafeed.com/newsroom/reports/rabobank-forecasts-demand-for-insect-protein-of-500000-tons-by-2030/.

The European insect protein market in 2023 reached EUR 218.63 million and is expected to grow to nearly EUR 1.67 billion by 2031, demonstrating an impressive compounded annual growth rate of 28.9% for that period. See, for example, https://www.databridgemarketresearch.com/reports/europe-insect-protein-market.

Insect excrement/leftovers (collectively called frass) introduction into circulation is subject to different regulations in different countries. For example, in the EU, it can be used in all crops, similarly to natural and mineral fertilizers, provided that the frass has been subjected to prior thermal treatment, the so-called heating at a minimum temperature of 70° C. for 60 minutes.

Hermetia illucens excrement has been used as a nutrient medium for fungi (note CN110012779 (A)). This reference employs fermentation of black fly insect feces, retention of residue squeeze, sterilization, inoculation of domestic strains of Streptomyces fine yellow (Streptomyces violascens) with laboratory bacteria codenamed LPF or heat-resistant laboratory bacteria Streptomyces (Streptomyces thermotolerans) codenamed HFM and then mixing the mass with other ingredients to obtain a medium for growing mushrooms. The excrement in this case was a semi-liquid excrement used on black flies wherein the fungi was inoculated with bacteria, and sterilized and fermented. Because of the complexity of this process, the costs for using this process are likely to be prohibitive, particularly if one attempts to scale the process up. That is, the process will be too cost prohibitive to be used on an industrial scale.

CN113854042 (A) discloses a method of planting and cultivating Morchella esculenta and circulating insects. Plastic bags with nutrients (leftovers) of Morchella esculenta are left and used as feed for the reproduction of unicorn beetle larvae. After the insects eat the feed, the remains are converted into feces, and subsequently the feces are used as auxiliary material for the cultivation of Morchella esculenta.

The method comprises the following steps: (1) collecting morchella residue; (2) feeding the unicorn beetle larvae with morchella remnants; (3) feces collection; (4) preparation of feeding bags according to the formula and (5) sowing Morchella esculenta, covering with soil, placing nutrient bags and carrying at harvest.
This process takes place in uncontrolled conditions with the use of wild insects and the frass does not undergo any controlled transformation processes. The uncontrolled conditions and the failure to allow the frass to undergo a controlled transformation process means that production of mushrooms and/or the insects cannot be controlled in a manner that makes growth on an industrial scale feasible. Moreover, because this process uses uncontrolled conditions and fails to allow the frass to undergo a controlled transformation process, the results of any mushrooms that are produced are likely to not meet the requisite food safety production standards as set by the various localities. When grown on an industrial scale, these food safety issues are likely to be exacerbated.

Accordingly, the present invention was developed with these shortcomings in mind.

### SUMMARY OF THE INVENTION

The object of the invention is the use of insect manure/fertilizer (frass) as well as protein and chitin obtained in the production of Coleoptera and Orthoptera insects in a controlled manner and/or on an industrial scale in the cultivation of edible and medicinal mushrooms (in particular the Agaricus bisporus). In an embodiment, the present invention relates to the breeding of insects and the cultivation of mushrooms in a closed loop system economy in order to reduce the carbon footprint and improve the efficiency and profitability of production.

In an embodiment, the present invention relates to an improved method of growth and cultivation of mushrooms and insects relative to the mushrooms and insects that are grown and cultivated more traditionally, for example, by using chicken and/or horse manure.

In an embodiment, the present invention relates to unexpectedly improved cultivation and growth of mushrooms using insect frass as an addition/replacement of low peat in casing for growing mushrooms. Moreover, in an embodiment, the present invention also relates to the use of protein and chitin as ingredients of mushroom feeders.

Furthermore, the present invention also relates to the unexpectedly superior effect of enriching the composition of insect frass produced in a closed loop system of insect and mushroom production, thereby improving the efficiency of mushroom cultivation.

### BRIEF DESCRIPTION OF THE SEVERAL VIEWS OF THE DRAWINGS

FIG. 1 shows the circularity of the mushroom-insect cycle in a closed loop system economy.

FIGS. 2-4 show the growth of different species of A. bisporus mycelium (A15, F1, Cayene Osadki, Mcorn and Gold Prot) under the same conditions on different media with and without the addition of frass.

FIG. 5 shows substrate in an autoclave.

FIG. 6 shows substrate ready for inoculation with the arboreal mushroom species.

FIG. 7 shows bales of substrate that have been inoculated with the arboreal mushroom species that are undergoing overgrowth.

FIGS. 8, 9, 10 show fruiting Lion's Mane (Hericium erinaceus) on substrate with the addition of insect frass

The results of these growth experiments are explained below.

### DETAILED DESCRIPTION OF THE INVENTION

The present invention relates to the use of insect fertilizer (frass) as well as protein and chitin obtained in the production of insects such as Coleoptera and Orthoptera on an industrial scale in the cultivation of edible and medicinal mushrooms (for example, the Agaricus bisporus mushrooms). In an embodiment, the present invention also relates to the breeding of insects and the cultivation of mushrooms in a closed loop system economy in order to reduce the carbon footprint and improve the efficiency and profitability of production.

In an embodiment, the frass of insects, such as using the frass of Coleoptera and Orthoptera species can be successfully used as an addition or replacement for chicken and horse manure in the production of substrates for mushrooms (for example, the Agaricus bisporus species). In an embodiment, using the frass of these insects leads to higher amounts of nitrogen and minerals relative to using the manure of animals (such as horse and/or chicken).

Using insects has several advantages such as the high content of protein, vitamins, elements and all essential amino acids, as well as their extraordinary ability to convert low-energy feed from agricultural and food industry by-products in a low-emission process into the highest quality protein, fat and chitin. Moreover, when these advantages are combined with a short fattening period, the use of insects may be placed at the forefront of the reconstructed food chain. Insect production also generates a by-product in the form of a high-quality natural fertilizer-frass in a dry and odorless form, with parameters comparable to mineral fertilizers.

Insect frass is a natural fertilizer, identical or superior to manure or chicken manure. The controlled production of insect frass as discussed herein allows the industrial production of mushrooms that can be produced with better nutrients at higher yields than methodologies used in the prior art.

Laboratory tests of the composition of frass of Alphitobius diaperinus species have demonstrated that the content of all key ingredients for mushroom cultivation occur at levels equal to or higher than chicken manure or horse manure. Accordingly, the present invention should be superior to methods that are used in the prior art (e.g., using chicken or horse manure). Tables 1-7 show the relative amounts of various fats, minerals, proteins, water, fiber and phosphorus and potassium and other components that can be obtained from the frass of Alphitobius diaperinus (from the insects both prior to and after being fed mushrooms). Tables 1-3 show the frass amounts of the various components from the insects without being fed mushrooms, and Tables 4-7 show the various component amounts in the frass of the insects after being fed mushrooms in their diet . . .

TABLE 1

| | Tested | | | | |
|---|---|---|---|---|---|
| No. | feature | Result ± uncertainty* | LOQ | Test method | |
| 1. | Total fat | 0.51 ± 0.03 g/100 g | 0.04 g/100 g | Extraction-weighing method according to | AN |
| | | | | Weibull-Stoldt, in accordance with PN-EN ISO | |
| | | | | 11085: 2015-10 | |
| 2. | Nitrogen | 3.05 ± 0.24 g/100 g | 0.018 g/100 g | Kjeldahl titration, in accordance with | AN |
| 3. | Raw protein | 19.06 ± 1.52 g/00 g | n/a | Commission Regulation (EC) No. 152/2009 of | AN |
| | | | | 27 Jan. 2009. annex III item C (Official Journal L. | |
| | | | | 54 of 26 Feb. 2009) excluding sampling. | |
| | | | | Crude protein content from calculations. | |
| | | | | (conversion factor 6.25) | |
| 4. | Water | 17.09 ± 0.56 g/100 g | 0.05 g/100 g | Gravimetric analysis, in accordance with | AN |
| 5. | Dry matter | 82.91 ± 0.56 g/100 g | n/a | Commission Regulation (EC) No. 152/2009 of | AN |
| | | | | 27 Jan. 2009. annex III item A (Official Journal. L | |
| | | | | 54 of 26 Feb. 2009) excluding sampling, drying | |
| | | | | temperature 130° C. Dry matter content from | |
| | | | | calculations | |
| 6. | Raw ash | 8.47 ± 0.37 g/100 g | 0.01 g/100 g | Gravimetric analysis, in accordance with | AN |
| | | | | Commission Regulation (EC) No. 152/2009 of | |
| | | | | 27 Jan. 2009. annex III item M (Journal U. L 54 of | |
| | | | | Feb. 26, 2009) excluding sampling, | |
| | | | | incineration temperature 550 C. | |
| 7. | Raw fiber | 16.68 ± 1.85 g/100 g | 0.19 g/100 g | PN ISO 5498: 1996 AOAC Official method | AN |
| | | | | 962.09 | |

*Expanded uncertainty for results in the quantitative range, for factor, extension k = 2 and confidence level of 95% (due to the fact that the laboratory does not take samples, this step is not included in the estimation of uncertainty);
LOQ—limit of quantification;
A—own accredited tests;
AN—own non-accredited research;
n/a—not applicable

TABLE 2

| | | Result ± | | | |
|---|---|---|---|---|---|
| No. | Tested feature | uncertainty* | LOQ | Test method | |
| 1 | aNDF-fiber | 47.08 ± | 1.20 g/100 g | ISO 16472: 2006 Application note | AN |
| | fraction | 4.10 g/100 g | | C. Gerhardt | |
| 2 | ADF-fiber | 27.28 ± | 1.24 g/100 g | PN ISO 13906: 2009 | AN |
| | fraction | 3.87 g/100 g | | AOAC Official method | |
| | | | | 973.18, Application note C. Gerhardt | |
| 3 | ADL-fiber | 7.98 ± | 1.23 g/100 g | PN ISO 13906: 2009 AOAC Official | AN |
| | fraction | 2.20 g/100 g | | Method 973: 18, Application note C. | |
| | | | | Gerhardt | |

*Expanded uncertainty for results in the quantitative range, for factor, extension k = 2 and confidence level of 95% (due to the fact that the laboratory does not take samples, this step is not included in the estimation of uncertainty); LOQ—limit of quantification; A—own accredited tests; AN—own non-accredited research; n/a—not applicable

TABLE 3

| No. | Tested feature | Result ± uncertainty* | Unit | Temperature | Test method | |
|---|---|---|---|---|---|---|
| 1 | Water activity | 0.712 ± 0.10 | a_{w} | 25° C. | Electrochemical | AN |
| | (A_{w}) | | | | water activity tests | |
| | | | | | according to PN- | |
| | | | | | ISO 21807: 2005 | |
| | | | | | and AOAC 978.18 | |

** Expanded uncertainty for results in the quantitative range, for the coefficient, extension k = 2 and confidence level 95% (due to the fact that the laboratory does not take samples, this stage was not included in the estimation of uncertainty); A—non-accredited tests. Scope of quantification: 0.064-0.999 a_{w}

TABLE 4

| | Tested | | | | |
|---|---|---|---|---|---|
| No. | feature | Result ± uncertainty* | LOQ | Test method | |
| 1. | Total fat | 0.49 ± 0.03 g/100 g | 0.04 g/100 g | Extraction-weighing method according to | AN |
| | | | | Weibull-Stoldt, in accordance with PN-EN ISO | |
| | | | | 11085: 2015-10 | |
| 2. | Nitrogen | 3.38 ± 0.27 g/100 g | 0.018 g/100 g | Kjeldahl titration, in accordance with | AN |
| 3. | Raw protein | 21.13 ± 1.69 g/00 g | n/a | Commission Regulation (EC) No. 152/2009 of | AN |
| | | | | 27 Jan. 2009. annex III item C (Official Journal L. | |
| | | | | 54 of 26 Feb. 2009) excluding sampling. | |
| | | | | Crude protein content from calculations. | |
| | | | | (conversion factor 6.25) | |
| 4. | Water | 15.89 ± 0.52 g/100 g | 0.05 g/100 g | Gravimetric analysis, in accordance with | AN |
| 5. | Dry matter | 84.11 ± 0.52 g/100 g | n/a | Commission Regulation (EC) No. 152/2009 of | AN |
| | | | | 27 Jan. 2009. annex III item A (Official Journal. L | |
| | | | | 54 of 26 Feb. 2009) excluding sampling, drying | |
| | | | | temperature 130° C. Dry matter content from | |
| | | | | calculations | |
| 6. | Raw ash | 8.98 ± 0.40 g/100 g | 0.01 g/100 g | Gravimetric analysis, in accordance with | AN |
| | | | | Commission Regulation (EC) No. 152/2009 of | |
| | | | | 27 Jan. 2009. annex III item M (Journal U. L 54 of | |
| | | | | Feb. 26, 2009) excluding sampling, | |
| | | | | incineration temperature 550 C. | |
| 7. | Raw fiber | 16.24 ± 1.80 g/100 g | 0.19 g/100 g | PN ISO 5498: 1996 AOAC Official method | AN |
| | | | | 962.09 | |

*Expanded uncertainty for results in the quantitative range, for factor, extension k = 2 and confidence level of 95% (due to the fact that the laboratory does not take samples, this step is not included in the estimation of uncertainty);
LOQ—limit of quantification;
A—own accredited tests;
AN—own non-accredited research;
n/a—not applicable

TABLE 5

| No. | Tested feature | Result ± uncertainty* | LOD/LOQ | Unit | Test method | |
|---|---|---|---|---|---|---|
| 1 | Potassium | 21415 ± 2142 | — | Mg/kg | PB-158/LF | PA |
| | | | | | ed. 7 of | |
| | | | | | Jul. 02, 2022 | |
| 2 | Total phosphorus | >10000 | — | Mg/kg | PB-158/LF | PA |
| | | | | | ed. 7 of | |
| | | | | | Jul. 02, 2022 | |

*Expanded uncertainty for results in the quantitative range, for factor, extension k = 2 and confidence level 95% (due to the fact that the laboratory does not take samples, this step is not included in the estimation of uncertainty);
LOD—limit of detection;
LOQ—limit of quantification;
PA—tests accredited by the subcontractor accreditation number AB 1095
Subcontracts are covered by the Nuscan Laboratory management system;
PN—tests not accredited by a subcontractor

TABLE 6

| | | Result ± | | | |
|---|---|---|---|---|---|
| No. | Tested feature | uncertainty* | LOQ | Test method | |
| 1 | aNDF-fiber | 46.36 ± | 1.20 g/100 g | ISO 16472: 2006 Application note | AN |
| | fraction | 4.03 g/100 g | | C. Gerhardt | |
| 2 | ADF-fiber | 27.76 ± | 1.24 g/100 g | PN ISO 13906: 2009 | AN |
| | fraction | 3.94 g/100 g | | AOAC Official method | |
| | | | | 973.18, Application note C. Gerhardt | |
| 3 | ADL-fiber | 8.97 ± | 1.23 g/100 g | PN ISO 13906: 2009 AOAC Official | AN |
| | fraction | 2.48 g/100 g | | Method 973: 18, Application note C. | |
| | | | | Gerhardt | |

*Expanded uncertainty for results in the quantitative range, for factor, extension k = 2 and confidence level of 95% (due to the fact that the laboratory does not take samples, this step is not included in the estimation of uncertainty); LOQ—limit of quantification; A—own accredited tests; AN—own non-accredited research; n/a—not applicable

TABLE 7

| No. | Tested feature | Result ± uncertainty* | Unit | Temperature | Test method | |
|---|---|---|---|---|---|---|
| 1 | Water activity | 0.702 ± 0.010 | a_{w} | 25° C. | Electrochemical water | AN |
| | (A_{w}) | | | | activity tests according | |
| | | | | | to PN-ISO 21807: 2005 | |
| | | | | | and AOAC 978.18 | |

** Expanded uncertainty for results in the quantitative range, for the coefficient, extension k = 2 and confidence level 95% (due to the fact that the laboratory does not take samples, this stage was not included in the estimation of uncertainty); A—non-accredited tests. Scope of quantification: 0.064-0.999 a_{w}

The chemical composition of this insect frass proves that not only will the frass enable one to attain the physical and chemical processes related to the formation and maturation of substrates for the production of mushrooms in phases I, II and III or bales for arboreal species, but the frass will ensure the ideal and/or appropriate supply and ratio of the key elements and/or ratio of nitrogen, phosphorous, and potassium (N:P:K). Moreover, the high protein, fat and chitin supply as generated will result in a form that is better absorbed by fungi, which the inventors believe should have a positive effect on the development of mycelium and subsequent yields.

In an embodiment, the present invention relates to the use of a hot phase in both the use of frass and the sanitization in the cultivation of mushrooms. In a variation of the invention, Coleoptera insect frass, due to its superior specific physical conditions and the components that can be derived from it, can be successfully used as an organic material that can serve as a substitute or an addition to the use of low peat casing.

In an embodiment, laboratory tests have been conducted with Coleoptera frass that have shown very good physical properties that make it ideally suited for use as a cover in order to obtain the appropriate level of humidity and create an ideal environment for the development of mycelium and mushroom fruiting bodies. The tested features of this frass are shown in table 2.

In addition, due to the relatively high content of protein and chitin found in the frass, the frass can also be used as a food additive to the phase III substrate or casing. As an additive, it can be used either in its raw form or after extraction from frass. Alternatively, it can be used using a combination of the two.

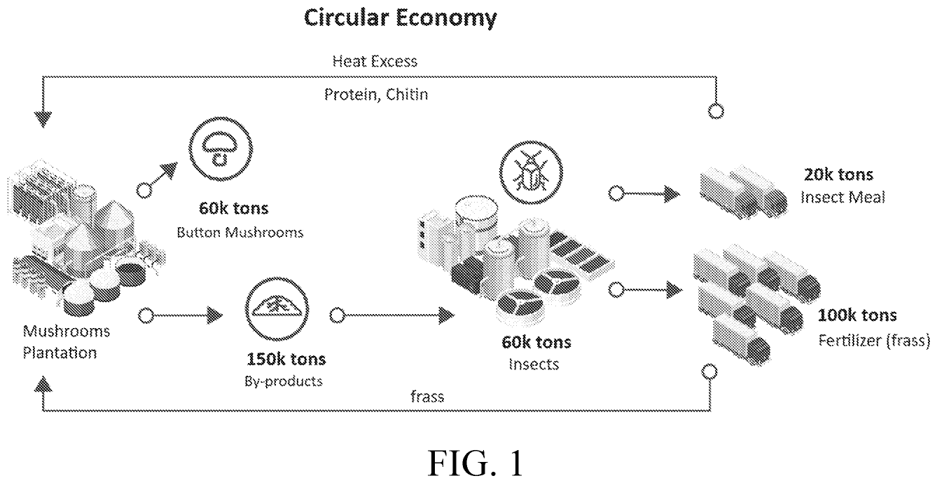

In connection with prior work done by the inventors of the present invention, one embodiment of the invention relates to the enrichment of insect meal with ingredients derived from biomass (substrates overgrown with mycelium, stems) after mushroom cultivation. In another embodiment of the present invention, mushrooms and insects can be produced synergistically together in a closed loop system economy. Thus, in a variation, the present invention relates to using a closed loop production of insects using Coleoptera (for example, A. diaperinus, T. molitor, Z. morio) or Orthoptera and edible and/or medicinal mushrooms (for example, A. bisporus, Pleurotus ostreatus, Pleurotus eryngii, Lentinula edodes, Agrocybe aegerita, Hericium erinaceus, Hypsizygus tessulatus, Flammulina velutipes). In an embodiment, the present invention comprises the cultivation of mushrooms using substrates for the production of mushrooms in which insect frass is used (1). It also relates to using frass as casing (2). Moreover, in an embodiment, the present invention relates to using frass as a food additive, wherein the frass comprises insect protein and insect chitin (3). In a variation, the present invention also relates to the production and use of substrates that are generated after mushroom cultivation for example, the production and use of spent mushroom substrate (SMS) or spent mushroom compost (SMC), stems, and/or damaged fruit bodies as feed/feed additive for insects to improve the quality of insect protein and frass as well as to fix/sequester carbon from SMS into insect bodies (4). In an embodiment, the present invention also relates to one or more of obtaining insect frass for re-use for the production of substrates, casings and feeders in mushroom cultivation (5), and/or extraction of protein and chitin from insects for use as feed additives in mushroom cultivation (6), as well as using metabolic heat of insects in order to warming up mushroom farms (7) The scheme of these various embodiments of the invention can be seen in FIG. 1.

In an embodiment and as shown in FIG. 1, the use of the mushroom insect frass cycle as shown should lead to a reduction in green-house gases for a plurality of reasons. First, the production of manure, which is typically used (and which can be replaced by Frass) is often accompanied by the production of methane, which is known to cause greater warming effects than the production of other greenhouse gases, such as carbon dioxide. Second, as one proceeds up the food chain (for example to animals such as chickens and/or horses/cows), there is a loss in the conversion of available nutrients/proteins/fats that can be used by, for example, humans. Accordingly, because the present invention is geared towards keeping the cycle at the lower end of the food chain (e.g., fungi and insects), one does not suffer the concomitant loss of available nutrients that is seen with the introduction of higher order organisms (such as birds and/or mammals). Third, when one introduces additional ingredients into a cycle, the transportation costs and other associated costs that are encountered with the introduction of these additional ingredients must be considered. To date, these costs tend to contribute to enhanced greenhouse gas emissions.

Additionally, emissions associated with heating mushroom crops, especially arboreal ones, with conventional systems based on burning fossil fuels generate huge quantities of greenhouse gases. Mushroom pickers in the USA and CA have to pay huge sums of carbon credits. Coleoptera and Diptera insects, in their larval stage, generate enormous amounts of metabolic and frictional heat, meaning that the excess heat must be dissipated. Due to differences in temperatures inside insect breeding halls and mushroom cultivation halls, the heat from the former can ideally be used for heating the latter, reducing the economic and ecological costs of cultivation.

Other advantages of the present invention are enumerated below.

### Advantages of the Invention

Replacing or adding chicken manure to insect frass brings many benefits. Because chicken manure can be eliminated or greatly reduced by the use of frass as a substitute or as an addition to chicken manure, the use of chickens can also be reduced. Poultry producers are using increasing amounts of antibiotics, hormones and other drugs to maintain declining poultry production. Moreover, the poultry industry uses large amounts of chemicals for disinfection purposes. The result of using these significant amounts of chemicals is that the chemicals are incorporated into the chickens and into their excrement. These chemicals that are found in chicken manure interfere with proper, and what is typically strictly standardized cultivation of mushrooms, thereby reducing yields. Furthermore, the presence of these chemicals prevents the organic/bio production of mushrooms. Because mushrooms are typically sold and marketed as being free of harmful chemical additives, the addition of these chemicals will adversely affect sales and marketing. In addition, in some regions there are regulations that prohibit or limit cage breeding, making the procurement of chicken manure more difficult, as the excrement is no longer localized in locations below cages.

The main benefit of using insect frass as an addition/replacement of low peat in casings is also beneficial for the environment. The massive destruction of non-renewable peatlands for mushroom cultivation is one of the great sins in the mushroom production industry. For example, in Poland, hundreds of thousands of cubic meters of peat are used annually for this purpose. The peat harvested for this use is typically limited to a single use, and its harvest irretrievably destroys tens/hundreds of hectares of peatlands, which are incredibly slow to recover (if they recover at all). Because of these problems, many countries have enacted bans on the use of peat in gardening. For example, Great Britain has introduced such a complete ban for several years, and other regions have instituted work on similar regulations (with rules and regulations being currently considered in the EU).

Another advantage of using insect frass in the casing is the direct supply of food additives such as protein and chitin in a more assimilable form, without the need for their fractionation. Accordingly, in an embodiment, the present invention relates to the direct incorporation of insect frass components into the cultivation of mushrooms in a form that is readily useable (without fractionation).

In an embodiment, the present invention also relates to a closed loop production system that is characterized by gigantic environmental benefits. In one variation, the closed loop production system significantly reduces greenhouse gas emissions associated with mushroom cultivation.

In an embodiment, production in a closed loop system means reducing the carbon footprint of transport, e.g., chicken and/or chicken manure, which, due to the fragmentation of the various industries and the huge demand of mushroom growers, must be transported over long distances. The emission of greenhouse gases related to the composting of used biomass after mushroom production is also reduced (for example, there is 750,000 tons per year in Poland, and 3.5 million tons per year in the EU, United States and Canada). Rather than composting the biomass after mushroom production, the biomass can be provided directly to the insects as a foodstuff. The FCR (feed conversion ratio) of insect breeding is about 4 kg of such biomass per 1 kg of insects, and the emission related to the production of insects is 1 g of greenhouse gases per 1 kg of insects. Therefore, the emission associated with the composting of mushroom industry waste is significantly higher. Composting organic biowaste releases significant amounts of CO₂, contributing to global warming. Assuming that SMS has a carbon content of 45% of its dry weight and that about 50% of this carbon is mineralized to CO₂ during composting, and considering that each gram of carbon will produce approximately 3.67 grams of CO₂, then composting 1 ton of SMS releases approximately 826 kg of CO₂.

Other methods such as anaerobic digestion (e.g. to produce biogas), incineration with energy recovery, or innovative industrial applications such as using spent mushroom substrate in packaging materials or other bioproducts are either not efficient considering the generated volume of SMS or, alternatively, it can release substantial amounts of CO₂. The scale of the problem can be illustrated by considering the amounts of mushrooms produced per annum. According to the GEPC (the European Mushroom Growers group) data, the production of Agaricus mushroom in the EU in 2022 was approximately 1.08M tons. This amount transfers approximately 4.3-5.4 M tons of SMS generated per annum in the EU, costing EU-based mushroom producers EUR 236-297M each year (assuming an average composting of SMS at 55 EUR/ton). Moreover, the related CO₂ emissions can reach up to 4.8M tons of CO₂ per annum. On a global scale, peer-reviewed articles reveal that the SMS of biowaste from the world's production of edible mushrooms is about 113.1 billion tons on a dry basis. Therefore, the relevant impact-related parameters need to be re-scaled using a factor of approximately 20×.

Mushroom production requires both a proper substrate and a casing layer, which is a mixture of peat moss and ground limestone. The casing material supports the growth of mushrooms. However, mushroom producers are not the only users of peat since it is also widely used as a major constituent of horticultural growing media. This is one area where a problem exists-peatlands are the largest natural terrestrial carbon store, while damaged peatlands are a major source of greenhouse gas emissions, responsible for almost 5% of global anthropogenic CO₂ emissions. As a result, several countries, such as the UK, Switzerland, and Norway, have proposed limiting or have already limited peat extraction and use, while other countries, including many other EU member states, will soon follow.

Not only does the closed loop production system have environmental benefits, the closed loop production system also provides huge economic benefits. In addition to the reduction in transportation costs alluded to above, the reduced transportation distances may result in carbon credits, and also reduce the costs associated with purchasing expensive protein and chitin feed supplements. Moreover, the closed loop production system also may provide additional revenue from the sale of insect meal and the surplus of frass.

In light of expected regulations on insect feed introduced in the EU, which prohibits the use of manure from farm animals, replacing manure with frass is the only and most efficient way to continue feeding insects with manure and mushroom biomass. Therefore, the present invention relates to replacing chicken manure with frass which is, at least in the EU, the only logical method of providing a closed loop system of insect and mushroom production. It allows the transfer and/or enrichment of insect meal with ingredients with medicinal and dietary values derived from mushroom biomass after mushroom cultivation.

Moreover, research conducted by the inventors in connection with this invention show unexpectedly improved properties wherein the frass of insects fed with biomass after mushroom cultivation is characterized by much better content parameters. That is, the ingredients essential in mushroom cultivation are present in a form much better absorbed by them. This combination of features shows the truly synergistic advancements that can be gained by adopting many of the measures detailed herein.

Additionally, frass obtained in the way described herein is an equally valuable addition/replacement to peat relative to insects that are traditionally fed.

### EXAMPLES SHOWING THE IMPLEMENTATION OF THE INVENTION

1. The larval form of insects of the Alphitobius diaperinus species was fed as needed with biomass from the cultivation of the Agaricus bisporus mushroom throughout the fattening period of 5 weeks. During harvesting, the larvae were mechanically separated from the frass. Frass was screened from uneaten feed residues on a sieve with a mesh size of 0.8 mm. The frass was thermally treated by heating at 70° C. for 60 minutes and examined. The results of laboratory tests gave the results shown in tables 4-7.

The frass obtained in this way was machine-mixed with Hajduk casing (a mixture of peat and chalk with a pH of 7.4 to 7.5 and a moisture content of 80-90%) in a volume ratio of 1:5 and 1:2 in two boxes with phase III substrate present on a total area 2×2.2 m². Subsequently, mycelium overgrowth in the casing with frass was observed whereas a reference cultivation carried out at the same time using Hajduk casing without the addition of frass did not show mycelium overgrowth. There were no differences between the applied frass doses and the reference crop. Cultivation was continued until the yield was 20 kg per m² in the first flush, 10 kg per m² in the second flush, and 6 kg per m² in the third flush. There were no significant differences in yield or in the amounts of components in the mushrooms between trials.

After each harvest, in boxes in which casing additives were used in the proportions of 1:5 and 1:2, the stems of mushrooms were manually removed from the casing, which, after appropriate processing, were fed to insects of the A. diaperinus species in the larval form. After the last flush, the substrate after cultivation (SMS) was mechanically separated from the casing, thermally disinfected with steam and, after appropriate treatment, fed to insects of the A. diaperinus species in the larval form. After the fattening period, the mature larvae were again separated from the frass, which, after appropriate processing, the frass was again added as an addition to the casing in the manner described above.

The above test was performed three times in a row in three different repetitions. The results of each repetition did not differ from each other by more than a statistical error. In an embodiment, “within statistical error means within one standard deviation of the mean.

Moreover, laboratory tests were performed to ascertain the effect of frass on the mycelium of various mushroom varieties. The results obtained show that the use of insect frass in phase I, phase II, and phase III in combination with the mushroom cultivation demonstrates that the closed loop process is not only feasible but results in very good production and yields for some of the tested species. In any event, because the insect frass and mushroom cultivation occur in a controlled manner, proof of concept of the invention has been confirmed and it is apparent that synergism, growth on an industrial scale, and a safe food product can be obtained that meets the rules/regulations of the localities.

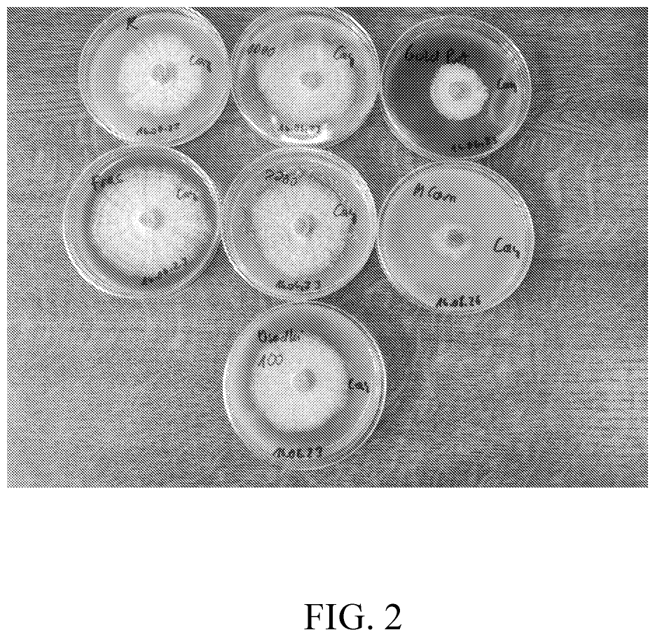

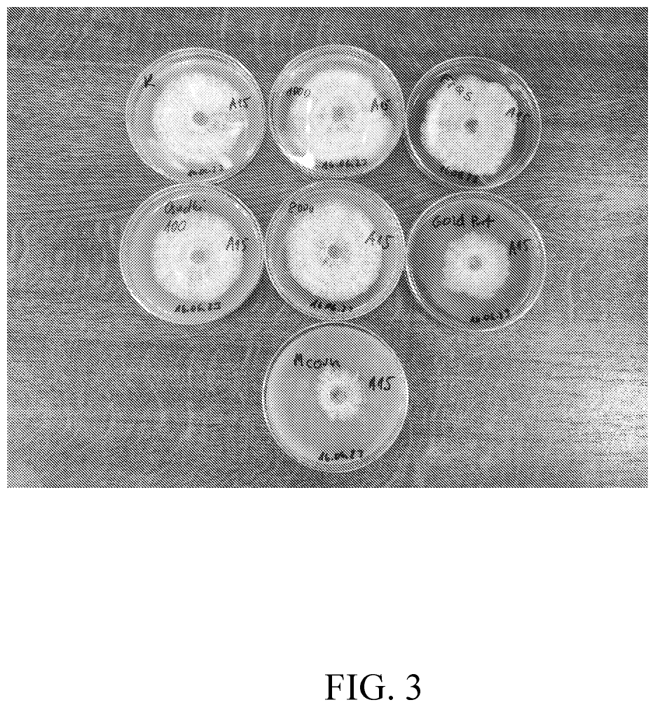

As shown in FIGS. 2 and 3, one can see the results of various comparative test preparations that were performed wherein wheat agar medium was used as a control and wheat agar medium with additives in the amount of 2% of product was used as the tested product. In the experiments, various mycelia were tested, with the A15, F1 and brown Cayene among the species being shown.

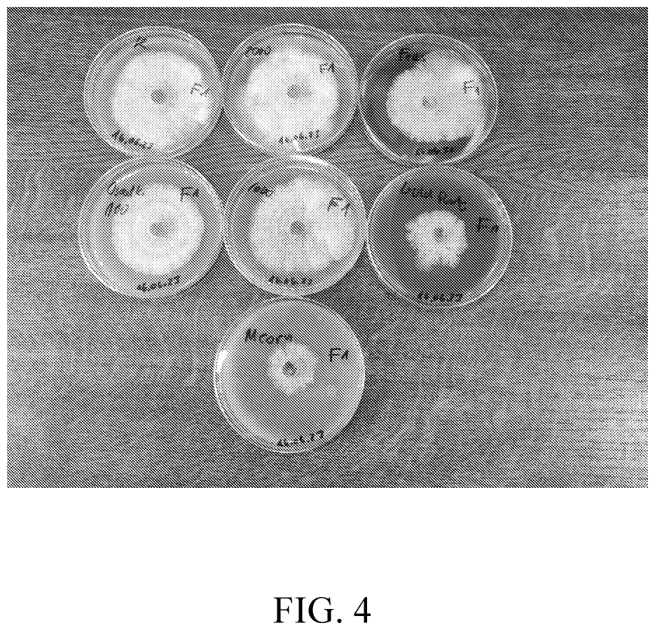

For the F1 strain (FIG. 4), the addition of insect frass did not show any clear growth acceleration. For the Cayene (FIG. 2) species, the addition of insect frass resulted in a slight acceleration of growth. But for A15, a clear growth acceleration was observed.

The results demonstrate that for at least several species of mushroom varieties, the addition of frass at the various phases of growth lead to accelerated growth. For other varieties, the addition of frass slows growth. However, for all varieties it works better than commonly used feeders such as Gold Prot or Mcorn.

Thus, the addition of insect frass can be used in a beneficial way to either enhance or inhibit growth. It should be apparent that if one is practicing polyculture wherein different varieties of mushroom are being grown together, the addition of insect frass, or withholding the insect frass can allow one to attain the preferential growth of whichever variety of mushroom one desires.

In an embodiment the present invention relates to a composition that comprises mushrooms that are being cultivated (meaning that they can be at any stage of the mushroom cultivation) in combination with insect frass. This composition may further comprise any one or more of the following ingredients: peat, chitin, other protein, manure (derived from any animal), straw, phosphogypsum, water, mushroom byproducts, insect byproducts, meal, other substrates, or any other ingredient mentioned herein. The insect frass may be derived from any of the species of insects disclosed herein and the mushrooms may be any species of mushroom described/disclosed herein.

In an embodiment the present invention relates to a process of cultivating mushrooms; said process comprising adding insect frass to mushrooms that are being cultivated, and allowing overgrowth to occur to generate cultivated mushrooms. In a variation, the insect frass is derived from Coleoptera and/or Orthoptera. In a variation, the process, further comprises adding chicken and/or horse manure. In a variation, the process has peat added. In a variation, insect frass is added to the peat. The addition of insect frass to the peat may happen prior to the peat being added to the mushrooms undergoing cultivation. In a variation, the process further comprises adding protein and/or chitin. In a variation, the protein and/or chitin may be extracted from insects. In a variation, the cultivated mushrooms and/or their byproducts are fed to insects to produce the insect frass. Thus, it should be apparent that there is a circular aspect to the invention wherein the insect frass is used to cultivate the mushrooms wherein the cultivated mushrooms can then be fed back to the insects to produce insect frass and that insect frass can be used to cultivate the mushrooms.

In a variation, the mushrooms in any embodiment of the invention are one or more members selected from the group consisting of Agaricus bisporus, Agaricus blazei, Pleurotus ostreatus, Pleurotus citrinopileatus, Pleurotus florida, Pleurotus pulmonarius, Pleurotus eryngii, Pleurotus djamor, Agrocybe aegerita, Hypsizygus ulmarius, Coprinus comatus, Ganoderma lucidum, Cordyceps militaris, Agaricus subrufescens, Stropharia rugosoannulata, Flammulina velutipes, Hypsizygus tessellatus, Hypsizygus marmoreus, Cyclocybe aegerita (cylindracea), Sparassis crispa, Hericium coralloides, Hericium erinaceus, Tuber borchii, Tuber indicum, Polyporus umbellatus, Grifola frondose, Laetiporus sulphureus, Ganoderma lucidum, Pholiota nameko, Kuehneromyces mutabilis, Volvariella volvacea, Hericium coralloides, Hericium erinaceus, Hericium abietis, Lentinula edodes, Auricularia polytricha, Grifola frondose, Psilocybe cyanescens, Psilocybe cubensis, Psilocybe semilanceata, Psilocybe mexicana, Psilocybe muscorumi, Stropharia cubensis, Trametes versicolor, Innonotus obliquus, Coriolus versicolor, Flammulina velutipes, and Phellinus linteus. It should be understood that the process/methodology disclosed herein can be used for either mushrooms that occur on the ground or arboreal mushrooms.

In a variation, the insect frass is derived from one or more members selected from the group consisting of Alphitobius diaperinus, Alphitobius laevigatus, Tenebrio molitor, Tenebrio obscurus, Tenebrio opacus, Zophobas atratus, Zophobas morio, Hermetia illucens, Musca domestica, Acheta domestica, Gryllodes sigillatus, Gryllus assimilis, Gryllus bimaculatus, Locusta migratoria, Schistocerca gregaria.

In an embodiment, the present invention relates to a process of cultivating mushrooms; said process comprising a first phase, a second phase and a third phase,
the first phase comprising procuring straw, phosphogypsum, water and optionally, liquid manure to generate a compost mixture and heating the compost mixture for at least 10 days with a temperature of at least 50° C. for at least three days and 80° C. for at least seven days;
the second phase comprising cooling the compost mixture and adding mushroom spores to the compost mixture, and subsequently pasteurizing and maturing the compost mixture comprising the mushroom spores at an elevated temperature to produce mushroom mycelium that are selected for growth in the third phase;
the third phase comprising placing the mushroom mycelium in overgrowth tunnels and cultivating the mushroom mycelium to produce cultivated mushrooms and/or their byproducts,
wherein insect frass is added in any or all of the first phase, the second phase, and the third phase. In a variation, the frass can be added at any amount.

In a variation, the insect frass is added in all of the first phase, the second phase and the third phase. In a variation, chicken and/or horse manure is added in the process. In a variation, peat is added in the first phase, the second phase or the third phase. In a variation, insect frass is added to the peat prior to the peat being used (for example, as casing). In a variation, the process further comprises adding protein and/or chitin. In a variation, the protein and/or chitin is extracted from insect meal.

In an embodiment, the insect frass is added in the second phase so that the insect frass is pasteurized. In a variation, the cultivated mushrooms and/or their byproducts are fed to insects to produce the insect frass.

In an embodiment, the present invention relates to a closed loop system comprising a synergistic relationship between cultivating mushrooms to produce mushrooms and/or their byproducts and insect frass produced during the growth of insects, the closed loop system comprising separating the insect frass from the insects, and using the insect frass as at least a fertilizer in the process of cultivating mushrooms and/or their byproducts, and using the mushrooms and/or their byproducts to feed the insects. In a variation, the closed loop system further comprises using the insect frass in combination with peat and/or chicken manure and/or horse manure to cultivate the mushrooms and/or their byproducts.

The closed loop system further comprises a) insect frass used in casings, b) insect frass used in combination with food additives that comprise protein and/or chitin extracted from the insects, c) insect frass used in combination with cultivated mushrooms, stems, and/or damaged fruiting bodies as fodder and/or a feed additive for the insects, or d) protein and/or chitin extracted from insect meal to be used as a food additive and/or e) heat from insect production that can be used for warming mushroom facilities.
2. In another embodiment, the present invention relates to using the substrate with the addition of frass for growing arboreal edible mushrooms in bales. In this example, three bale versions were tested, each bale differing in the relative content of insect manure.
   Substrates were tested that had the compositions shown in table 8.

TABLE 8

| Substrate 1 | Substrate 2 | Substrate 3 |
|---|---|---|
| Beech sawdust 95% | Beech sawdust 85% | Beech sawdust 75% |
| Frass 5% | Frass 15% | Frass 25% |
| With a humidity | With a humidity | With a humidity |
| of 55% | of 55% | of 55% |

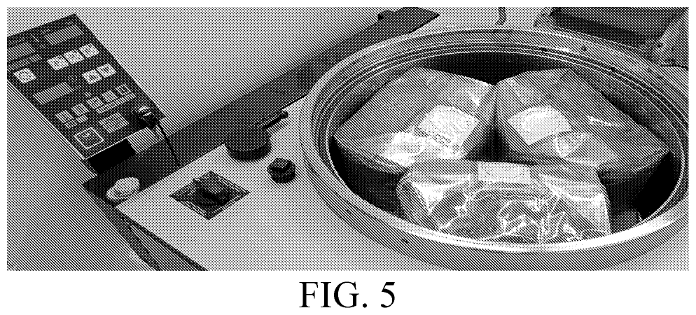

The various substrates were sterilized 15 pieces at a time in a H+P Varioklav 400E autoclave at a temperature of 121° C. for 90 minutes. After sterilization, the substrates were cooled in a clean room for 12 hours until they reached ambient temperature (˜21° C.). FIG. 5 shows an image of the substrate (containing insect frass and beech sawdust in an autoclave).

The results of these growth experiments are explained below.

30 mm size (Sakato MP30 mm) bags equipped with filters with a pore diameter of 5 μm were used for testing.

Lion's Mane Hericium erinaceus vaccination material was prepared 2 weeks earlier in liquid culture aerated with sterile air (Millipore 0.22 μm filters were used to purify the air).

After overgrowth of the liquid culture containing the Lion's Mane Hericium erinaceus, the various media were inoculated/vaccinated with this liquid inoculation material.

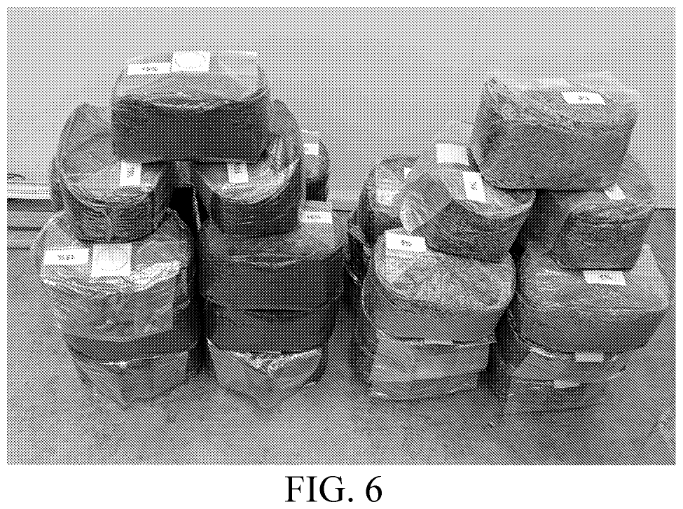

FIG. 6 shows substrate ready for inoculation with the arboreal mushroom species.

Vaccination was performed on each of 7 bags/bales of each substrate per species, and one bag/bale was left as a sterility control.

During mycelium overgrowth, mycelial growth was observed and the following summarizes the results that were attained:
1. The 5% medium demonstrated the best (most rapid) growth of all 3 versions.
2. The substrate containing 25% frass had an almost comparable growth rate.
3. The 15% frass medium showed the slowest growth

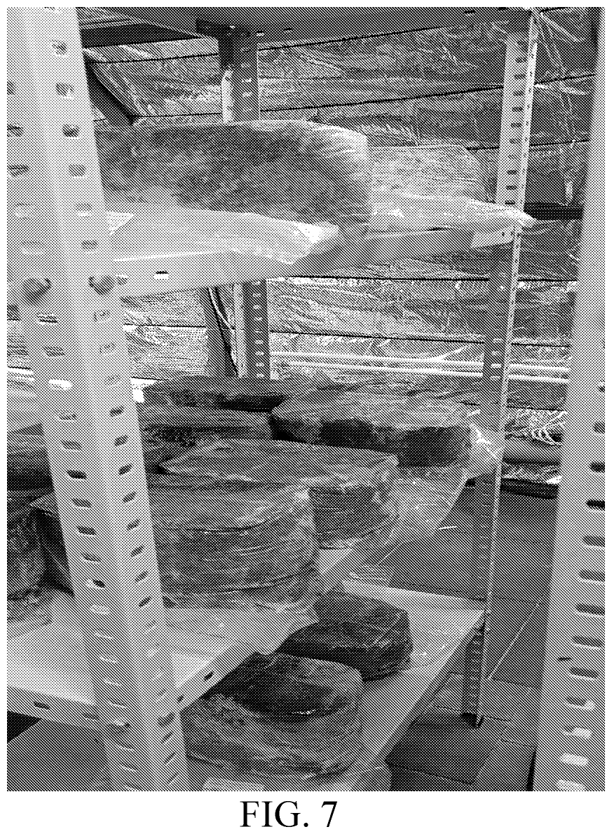

Mycelium growth rates were demonstrated to be more rapid at 5% and 25%. FIG. 7 shows bales of substrate that have been inoculated with the arboreal mushroom species that are undergoing overgrowth.

The bags were then placed in a climate-controlled tent to allow the fruiting bodies to grow. The climate controlled tent had a temperature of 19-21° C., a relative humidity of 85-91% and a CO₂ content of 700-800 ppm.

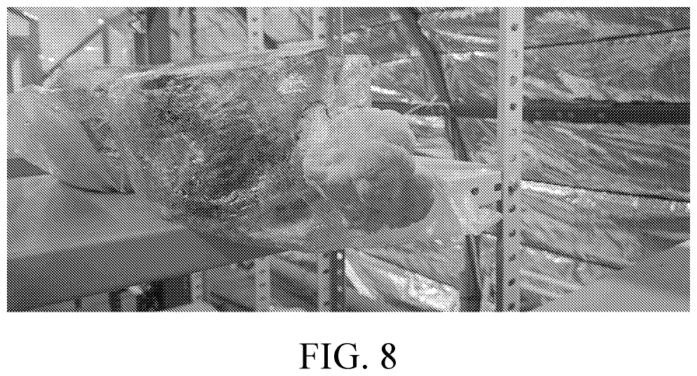

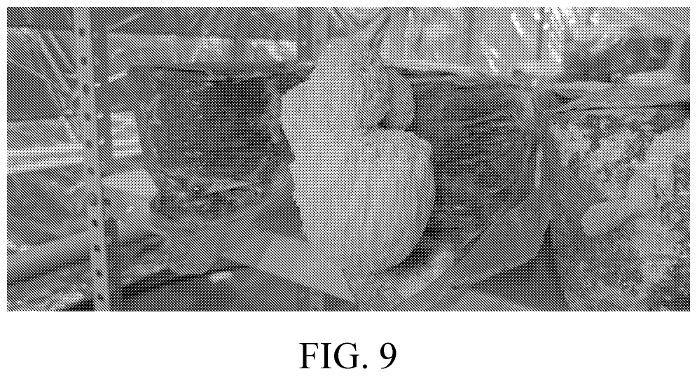

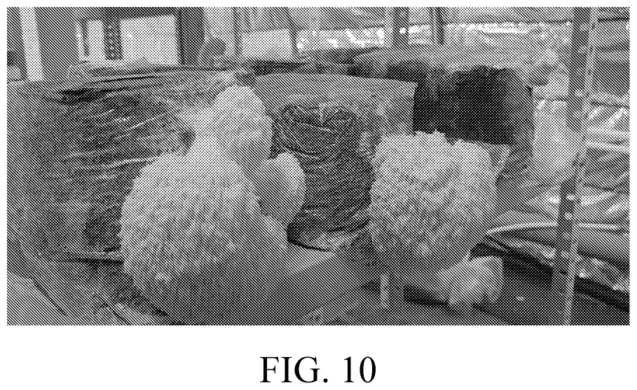

FIGS. 8, 9, and 10 show fruiting Lion's Mane (Hericium erinaceus) on substrate with the addition of insect frass.

During the harvest, the highest yields were recorded in the substrate with 25% of insect excrements added. The harvest with this addition was approximately 20% larger than the standard one with 45% organic bran content, and the fruit bodies were more compact and healthier. Taking into account that frass is an organic product and its price is half of the price of organic bran, it is economically justified to use it as an addition to mixtures or completely replace it in substrates for growing arboreal mushrooms.

More research is needed to determine the biosynthesis of active compounds or to detect changes in the composition and physico-chemical characteristics of the fruit bodies themselves.

All references recited herein are incorporated by reference in their entireties for all purposes.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

All references cited herein are incorporated by reference in their entireties.

We claim:

1. A process of cultivating mushrooms; said process comprising adding insect frass to substrate and/or mushrooms that are being cultivated, in order to allow mycelium overgrowth and/or to produce fruiting bodies of cultivated mushrooms.

2. The process of claim 1, wherein the insect frass is from Coleoptera and/or Orthoptera.

3. The process of claim 1, further comprising adding chicken and/or horse manure.

4. The process of claim 1, wherein peat is added.

5. The process of claim 4, wherein insect frass is added to the peat.

6. The process of claim 1, further comprising adding protein and/or chitin.

7. The process of claim 6, wherein the protein and/or chitin is extracted from insects.

8. The process of claim 1, wherein the cultivated mushrooms and/or their byproducts are fed to insects to produce the insect frass.

9. The process of claim 1, wherein the mushrooms are one or more members selected from the group consisting of Agaricus bisporus, Agaricus blazei, Pleurotus ostreatus, Pleurotus citrinopileatus, Pleurotus florida, Pleurotus pulmonarius, Pleurotus eryngii, Pleurotus djamor, Agrocybe aegerita, Hypsizygus ulmarius, Coprimis comatus, Ganoderma lucidum, Cordyceps militaris, Agaricus subrufescens, Stropharia rugosoanmilata, Flammulina velutipes, Hypsizygus tessellatus, Hypsizygus marmoreus, Cyclocybe aegerita (cylindracea), Sparassis crispa, Hericium coralloides, Hericium erinaceus, Tuber borchii, Tuber indicum, Polyporus umbellatus, Grifola frondose, Laetiporus sulphureus, Ganoderma lucidum, Pholiota nameko, Kuehneromyces mutabilis, Volvariella volvacea, Hericium coralloides, Hericium erinaceus, Hericium abietis, Lentimila edodes, Auricularia polytricha, Grifola frondose, Psilocybe cyanescens, Psilocybe cubensis, Psilocybe semilanceata, Psilocybe mexicana, Psilocybe muscorumi, Stropharia cubensis, Trametes versicolor, Innonotus obliquus, Coriolus versicolor, Flammulina velutipes, and Phellinus linteus.

10. The process of claim 1, wherein the insect frass is derived from one or more members selected from the group consisting of Alphitobius diaperimis, Alphitobius laevigatus, Tenebrio molitor, Tenebrio obscurus, Tenebrio opacus, Zophobas atratus, Zophobas morio, Hermetia illucens, Musca domestica, Acheta domestica, Gryllodes sigillatus, Gryllus assimilis, Gryllus bimaculatus, Locusta migratoria, Schistocerca gregaria.

11. A closed loop system comprising a synergistic relationship between cultivating mushrooms to produce mushrooms and/or their byproducts and insect growth and insect frass produced during the growth of insects, the closed loop system comprising separating the insect frass from the insects, and using the insect frass as at least a fertilizer in the process of cultivating mushrooms and/or their byproducts, and using the mushrooms and/or their byproducts to feed the insects.

12. The closed loop system of claim 11, further comprising using the insect frass in combination with peat and/or chicken manure and/or horse manure to cultivate the mushrooms and/or their byproducts.

13. The closed loop system of claim 11, wherein the system further comprises a) insect frass used in casings, b) insect frass used in combination with food additives that comprise protein and/or chitin extracted from the insects, c) insect frass used in combination with cultivated mushrooms, stems, and/or damaged fruiting bodies as fodder and/or a feed additive for the insects, or d) protein and/or chitin extracted from insect meal to be used as a food additive.

14. The closed loop system of claim 11, wherein the closed loop system achieves at least one of; a) reducing greenhouse gases relative to mushroom cultivation that does not use the insect frass and insect feed from the by-products of mushroom cultivation, and b) recovering heat from metabolic processes during the insect growth to use as a heat source to heat farms in which mushroom cultivation occurs.

15. The closed loop system of claim 14, wherein the closed loop system achieves both a) reducing greenhouse gases relative to mushroom cultivation that does not use the insect frass and insect feed from the by-products of mushroom cultivation, and b) recovering heat from metabolic processes during the insect growth to use as a heat source to heat farms in which mushroom cultivation occurs.

16. A process for growing arboreal mushrooms, said process comprising:
obtaining a substrate that comprises insect frass
heating the substrate to sterilize the substrate;
obtaining a liquid vaccination material that comprises an arboreal mushroom mycelium;
placing the substrate in bales or logs and inoculating the substrate with liquid vaccination material and allowing overgrowth to occur in the bales or logs.

17. The process of claim 16, wherein the substrate further comprises one or more of shavings from deciduous trees, straw, fruit tree sawdust, and cereals, wherein the cereals are one or more members selected from the group consisting of wheat, corn, soy hull, sorghum, and/or their milling products.

18. The process of claim 16, wherein the milling products are one or more members selected from the group consisting of bran, nut shells, hemp straw, coconut, vermiculite and cardboard and/or wherein the substrate further comprises insect frass.

19. The process of claim 17, wherein the milling products are one or more members selected from the group consisting of bran, nut shells, hemp straw, coconut, vermiculite and cardboard and/or wherein the substrate further comprises insect frass.

20. The process of claim 18, wherein the arboreal mushrooms are one or more members selected from the group consisting of Lion's Mane Hericium erinaceus, Pleurotus ostreatus, Pleurotus eryngii, Lentimia edodes, Agrocybe aegerita, Hypsizygus tessulatus, and Flammulina velutipes, wherein the insect frass is present in an amount from 5% to 25% by weight of the substrate.
